(19) United States
(12) Patent Application Publication
(10) Pub. No.: US 2018/0303400 A1
(43) Pub. Date: Oct. 25, 2018

(54) AUTOMATED STROKE DETECTION AND TREATMENT SUPPORT SYSTEM

(71) Applicant: Qspin, LLC, Las Vegas, NV (US)

(72) Inventor: Evan Calabrese, San Francisco, CA (US)

(21) Appl. No.: 15/492,206

(22) Filed: Apr. 20, 2017

(51) Int. Cl. A61B 5/00; A61B 5/055; A61B 6/03; A61B 6/00

(52) U.S. Cl. CPC A61B 5/4076; A61B 5/4064; A61B 6/501; A61B 5/055; A61B 6/032; A61B 5/7271

(57) ABSTRACT

A automated stroke detection and treatment support system can include: a brain scan analyzer for determining a stroke diagnosis in response to a brain scan of a patient, the stroke diagnosis including an indication of whether or not the brain scan indicates a possible stroke and a set of parameters pertaining to the possible stroke; and a treatment planner for generating a treatment report for aiding a physician treating the patient in response to the stroke diagnosis.

### BACKGROUND

A patient who suffers a stroke can be taken to a hospital, clinic, or other treatment center. A treating physician, e.g., an emergency room physician, at a treatment center can order a brain scan on a patient who suffers a stroke. The brain scan can be provided to a specialist in stroke diagnosis, e.g., a radiologist, a brain surgeon, etc. A specialist can communicate a stroke diagnosis back to the treating physician. The treating physician can then make arrangements with other staff for treating the patient based on the stroke diagnosis.

Time can be of the essence when determining whether or not a patient has suffered a stroke and in treating a stroke. Human emotions, time pressures, miscommunications among staff, etc., can lead to delays and errors in stroke treatment. Uncertainty surrounding the existence of a stroke, or the type of stroke, e.g., ischemic or hemorrhagic, can delay appropriate treatment or result in potentially harmful unnecessary treatments. Delays and errors in stroke treatment can cause long-term physical disability, mental disability, or death of the patient who suffers the stroke.

### SUMMARY

In general, in one aspect, the invention relates to an automated stroke detection and treatment support system. The system can include: a brain scan analyzer for determining a stroke diagnosis in response to a brain scan of a patient, the stroke diagnosis including an indication of whether or not the brain scan indicates a possible stroke and a set of parameters pertaining to the possible stroke; and a treatment planner for generating a treatment report in response to the stroke diagnosis, the treatment report for aiding a physician treating the patient.

In general, in another aspect, the invention relates to a method for automated stroke detection and treatment support. The method can include: determining a stroke diagnosis by performing an automated analysis of a brain scan of a patient such that the stroke diagnosis includes an indication of whether or not the brain scan indicates a possible stroke and further includes a set of parameters pertaining to the possible stroke; and generating a treatment report in response to the stroke diagnosis, the treatment report for aiding a physician treating the patient.

Other aspects of the invention will be apparent from the following description and the appended claims.

### BRIEF DESCRIPTION OF THE DRAWINGS

Embodiments of the present invention are illustrated by way of example, and not by way of limitation, in the figures of the accompanying drawings and in which like reference numerals refer to similar elements.

FIG. 1 shows an automated stroke detection and treatment support system in one or more embodiments.

FIG. 2 shows an embodiment of an automated stroke detection and treatment support system that includes a local knowledge store.

FIG. 3 shows an example of how information can be arranged in a knowledge store of an automated stroke detection and treatment support system.

FIG. 4 shows an embodiment in which an automated stroke detection and treatment support system is included as part of a medical imaging facility.

FIG. 5 shows how a physician can use a mobile computing device to access a treatment report generated by an automated stroke detection and treatment support system.

FIG. 6 shows how a specialist can provide a feedback pertaining to an automated stroke detection and treatment support system.

FIG. 7 shows how a brain scan analyzer of an automated stroke detection and treatment support system can locate a possible stroke in a brain scan.

FIG. 8 illustrates a method for automated stroke detection and treatment support in one or more embodiments.

FIG. 9 illustrates a computing system upon which portions of an automated stroke detection and treatment support system can be implemented.

### DETAILED DESCRIPTION

Reference will now be made in detail to the various embodiments of the present disclosure, examples of which are illustrated in the accompanying drawings. Like elements in the various figures are denoted by like reference numerals for consistency. While described in conjunction with these embodiments, it will be understood that they are not intended to limit the disclosure to these embodiments. On the contrary, the disclosure is intended to cover alternatives, modifications and equivalents, which may be included within the spirit and scope of the disclosure as defined by the appended claims. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, have not been described in detail so as not to unnecessarily obscure aspects of the present disclosure.

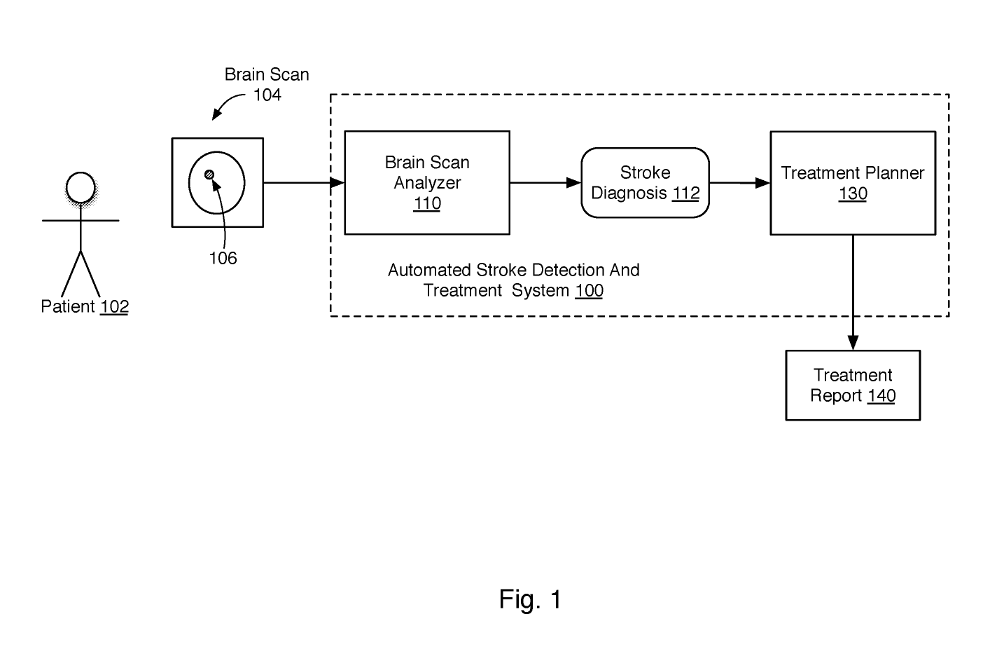

FIG. 1 shows an automated stroke detection and treatment support system 100 in one or more embodiments. The automated stroke detection and treatment support system 100 includes a brain scan analyzer 110 for determining a stroke diagnosis 112 in response to a brain scan 104 of a patient 102. The stroke diagnosis 112 in one or more embodiments includes an indication of whether or not the brain scan 104 indicates a possible stroke 106 and a set of parameters pertaining to the possible stroke 106.

The parameters in the stroke diagnosis 112 can include an identification of location, a brain region, etc., of the possible stroke 106. The parameters in the stroke diagnosis 112 can identify a location using terminology commonly employed in medical literature so that it is useful to a physician, clinician, etc., who is responsible for treating the patient 102. For example, the parameters in the stroke diagnosis 112 can identify “left frontal lobe primary motor cortex”, for the location of the possible stroke 106.

The parameters in the stroke diagnosis 112 can include a size, e.g., a volume, of the possible stroke 106. The parameters in the stroke diagnosis 112 can specify a vascular territory of the possible stroke 106. The parameters in the stroke diagnosis 112 can specify a distribution of the possible stroke 106, e.g., a singular stroke, multiple strokes, all in one vascular territory, in multiple different territories, etc. The parameters in the stroke diagnosis 112 can specify an anatomic region, e.g., an anatomic region defined by gross or microscopic anatomy. The parameters in the stroke diagnosis 112 can specify a vascular region, e.g., a vascular region defined by blood flow. The parameters in the stroke diagnosis 112 can specify a functional region, e.g., a functional region defined by brain activation patterns.

The automated stroke detection and treatment support system 100 includes a treatment planner 130 for generating a treatment report 140 in response to the stroke diagnosis 112. The treatment report 140 can be generated to aid a physician in treating the patient 102 for the possible stroke 106.

The treatment planner 130 can adapt the treatment report 140 based on any of the parameters in the stroke diagnosis 112. For example, if the stroke diagnosis 112 indicates that the possible stroke 106 is in the “left frontal lobe”, e.g., left frontal lobe, primary motor cortex, e.g., supplied by the left middle cerebral artery, then the treatment planner 130 adapts the treatment report 140 to aid a physician in treating the possible stroke 106 in that location. In this way, treatments identified in the treatment report 140 can be adapted based on vascular territory, and symptoms identified in the treatment report 140 can be adapted based on functional subdivisions of the brain.

In one or more embodiments, the treatment report 140 identifies a set of expected symptoms for clinical confirmation of the possible stroke 106. For example, if the stroke diagnosis 112 indicates that the possible stroke 106 is in the left frontal lobe, the expected symptoms identified in the treatment report 140 can include, e.g., an inability of a patent to move their right arm.

In one or more embodiments, the treatment report 140 identifies a recommended treatment for the possible stroke 106. For example, if the stroke diagnosis 112 indicates that the possible stroke 106 is in the “left middle cerebral artery territory”, the treatment report 140 can indicate current recommended treatment for strokes in that area. The treatment report 140 can include, e.g., procedures for using clot-busting, i.e., thrombolytic, medications, catheter-directed surgical procedures for clot removal, recommended medications, surgical devices, etc., that have proven to be effective for strokes in the left middle cerebral artery territory.

In one or more embodiments, the treatment report 140 can include a set of text. For example, the treatment report 140 can include a text description of the location of the possible stroke 106, e.g., “left frontal lobe primary motor cortex”. The treatment report 140 can include a text description of expected symptoms for clinical confirmation of the possible stroke 106. The treatment report 140 can include a text description of a recommended treatment for the possible stroke 106. Text in the treatment report 140 can be embodied in hardcopy printout, an HTML document, a text file, a digital printing document, e.g., PDF, etc.

In one or more embodiments, the treatment report 140 can include one or more illustrations. For example, the treatment report 140 can include an illustration of the possible stroke 106. The treatment report 140 can include illustrations of the expected symptoms for clinical confirmation of the possible stroke 106 and how to test for the expected symptoms. The treatment report 140 can include illustrations of recommended treatments for the possible stroke 106. Illustrations in the treatment report 140 can be embodied in hardcopy printout, web pages, digital documents, e.g., PDF, digital documents adapted for presentations, etc. Illustrations in the treatment report 140 can be audiovisual, e.g., videos, animations, etc. The treatment report 140 can include detailed tutorials and or videos illustrating how to elicit specific expected physical exam findings, in case the treating physician is not familiar with eliciting these exam findings.

In one or more embodiments, the treatment report 140 can include one or more uniform resource locators (URLs). For example, the treatment report 140 can include URLs for accessing text, illustrations, etc., pertaining to the size, location, and appearance of the possible stroke 106. The treatment report 140 can include URLs for accessing text, illustrations, videos, etc., pertaining to the expected symptoms for clinical confirmation of the possible stroke 106. The treatment report 140 can include URLs for accessing text, illustrations, videos, etc., pertaining to a recommended treatment for the possible stroke 106.

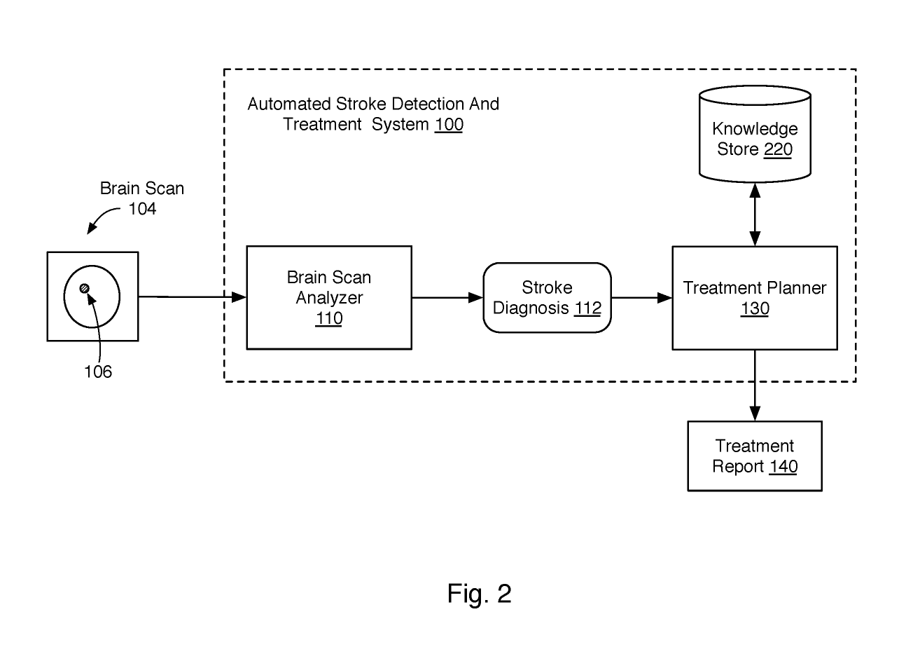

FIG. 2 shows an embodiment of the automated stroke detection and treatment support system 100 that includes a knowledge store 220. The knowledge store 220 can hold information that enables the treatment planner 130 to generate the treatment report 140 in response to the stroke diagnosis 112. For example, the knowledge store 220 can hold text, illustrations, URLs, videos, etc., pertaining to stroke symptoms and treatments for a variety of possible strokes.

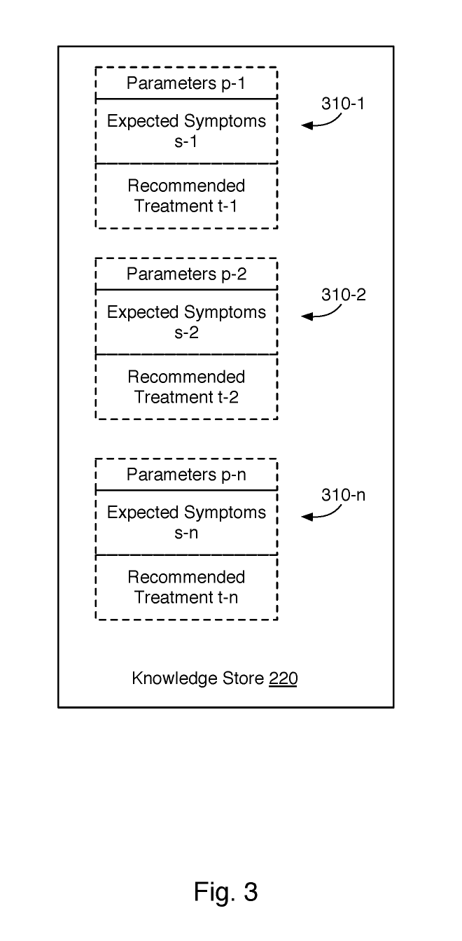

FIG. 3 is an example of how information can be arranged in the knowledge store 220 in one or more embodiments. The knowledge store 220 can hold multiple sets of treatment information 310-1 through 310-n. Each set of treatment information 310-1 through 310-n includes a corresponding set of parameters p-1 through p-n, a corresponding set of expected symptoms s-1 through s-n, and a corresponding recommended treatment t-1 through t-n.

The treatment planner 130 can generate the treatment report 140 by searching the knowledge store 220 for information pertinent to the stroke diagnosis 112. For example, the treatment planner 130 can match the parameters of the possible stroke 106 that are included in the stroke diagnosis 112 to each set of parameters p-1 through p-n and then extract the corresponding expected symptoms s-1 through s-n and the corresponding recommended treatments t-1 through t-n.

For example, the parameters p-1 can indicate a possible stroke in the “left frontal lobe” and the expected symptoms s-1 can include, e.g., an inability of a patent to move their right arm, and the recommended treatment t-1 can specify, e.g., clot-busting medications or an embolectomy, recommended medications, surgical procedures, surgical devices, etc., that have proven to be effective for treating strokes in the left frontal lobe.

The information 310-1 through 310-n can be derived from currently available scientific literature, professional society recommendations, government agency recommendations and rules, etc. The information 310-1 through 310-n can be updated at any time to reflect the latest scientific evidence, rules and regulations, and as new drugs, therapies, procedures, and medical devices become available. The information 310-1 through 310-n can include any combination of text, illustrations audiovisual material, URLs, articles, summaries, etc.

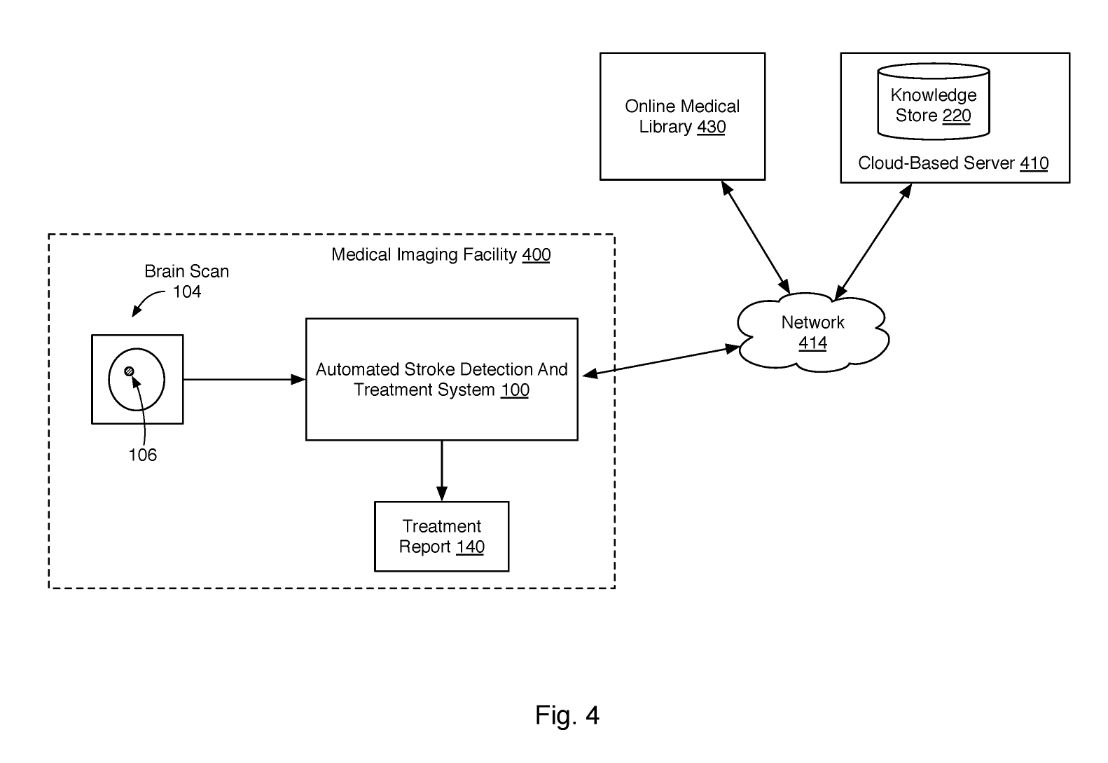

FIG. 4 shows an embodiment in which the automated stroke detection and treatment support system 100 is included as part of a medical imaging facility 400. The medical imaging facility 400 can be a computed tomography (CT) or magnetic resonance imaging (MRI) facility of a hospital, clinic, emergency facility, or other venue for treating patients, e.g., the patient 102. The automated stroke detection and treatment support system 100 can be implemented on code using the same computing resources in the medical imaging facility 400 used for generating the brain scan 104 from raw CT or MRI data.

In one or more embodiments, the knowledge store 220 is provided in a cloud-based server 410. The automated stroke detection and treatment support system 100 can access the cloud-based server 410 via a network 414.

In one or more embodiments, one or more of the URLs in the treatment report 140 can target resources in an online medical library 430. The resources of the online medical library 430 can include resources pertaining to stroke symptoms, recommended procedures, scientific publications, postings by specialists, etc.

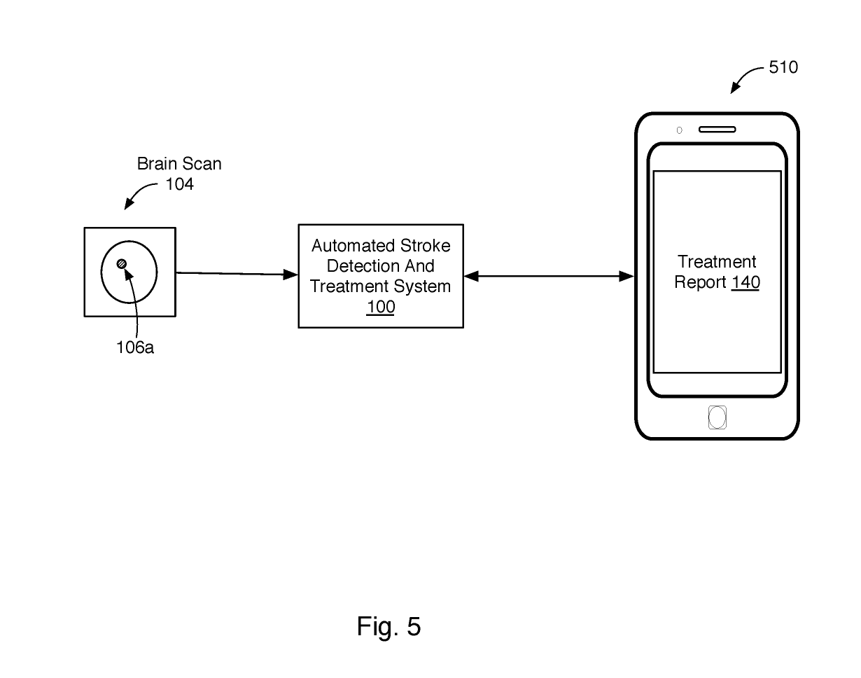

FIG. 5 shows how the automated stroke detection and treatment support system 100 can include an interface that enables a physician responsible for treating the patient 102 to access the treatment report 140 using a computing device 510 in one or more embodiments. For example, the computing device 510 can be a personal computer, a tablet, smartphone, wearable, specialized medical information device, etc. The automated stroke detection and treatment support system 100 can generate a web page for providing access to the treatment report 140 using web protocols. In some embodiments, the computing device 510 can run a mobile app adapted for accessing the automated stroke detection and treatment support system 100 and obtaining the treatment report 140.

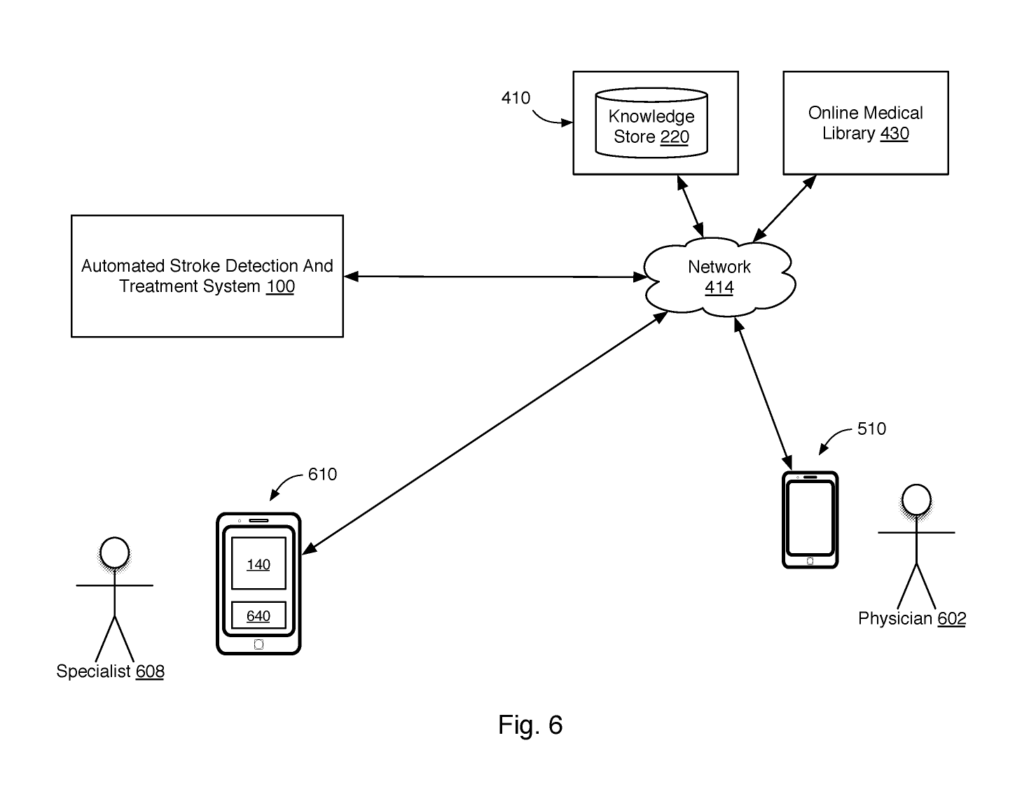

FIG. 6 shows how a physician 602 who is responsible for the immediate treatment of the patient 102 can access the treatment report 140 using the computing device 510 and how a specialist 608 can provide a feedback pertaining to the treatment report 140 using a computing device 610. The physician 602 can be, e.g., a treating physician, an emergency room physician, etc. The specialist 608 can be, e.g. a radiologist, a brain surgeon, etc.

The automated stroke detection and treatment support system 100 can generate a feedback interface 640 on the computing device 610 that enables the specialist 608 to provide feedback pertaining to the stroke diagnosis 112 as reflected in the treatment report 140. An example of feedback via the feedback interface 640 is “Yes” if the specialist 608 agrees with the diagnosis in the treatment report 140 or “No” if the specialist 608 does not agree. For example, the specialist 608 can view the brain scan 104 and perform their own analysis and then indicate Yes/No via the feedback interface 640. The feedback provided by the specialist 608 can be used as a data point to refine the brain scan analyzer 110 in the automated stroke detection and treatment support system 100.

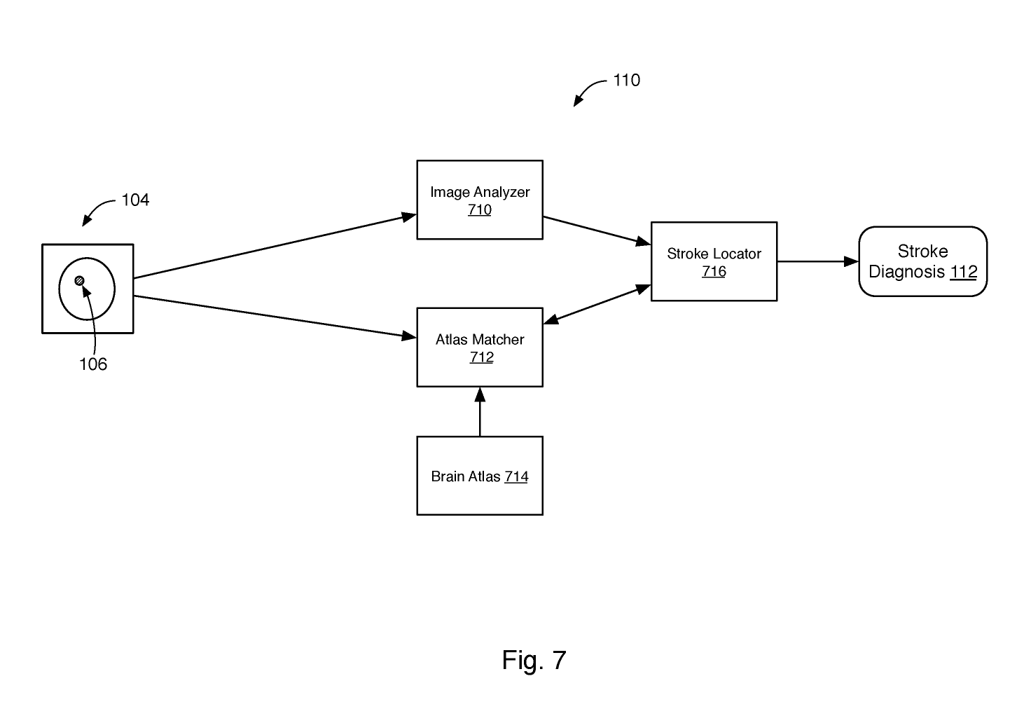

FIG. 7 shows how the brain scan analyzer 110 can identify the possible stroke 106 in response to the brain scan 104 in one or more embodiments. The brain scan 104 can be a three-dimensional array of pixels each having a corresponding intensity such that the three-dimensional array of pixels provides a 3D visual representation of the brain of the patient 102 including a depiction of the possible stroke 106. The brain scan 104 can be derived from computed tomography (CT) scans, magnetic resonance imaging (MM) scans, etc.

The brain scan analyzer 110 includes an image analyzer 710 that can determine which of the pixels of the brain scan 104 have an inordinately high intensity value or an inordinately low intensity value indicating unusually bright or dark areas of the brain of the patient 102. The image analyzer 710 can determine which of the pixels of the brain scan 104 have an abnormal or asymmetric pattern of pixel intensity.

For example, the image analyzer 710 can generate a histogram of pixel intensity values in the brain scan 104, detect inordinately high intensity values or inordinately low intensity values in the histogram, and determine which pixels in the brain scan 104 correspond to the inordinately high intensity values or inordinately low intensity values in the histogram. The 3D coordinates of the matching pixels in a coordinate space of the brain scan 104 provide a location of the possible stroke 106 in the coordinate space of the brain scan 104 or other coordinate spaces such as the coordinate space of the brain atlas, or standard anatomic coordinate systems used in the medical field.

The brain scan analyzer 110 includes a brain atlas 714 that provides a 3D visual representation of a reference brain including annotations that are pertinent to treatment information widely available in the scientific literature. For example, 3D regions of pixels in the brain atlas 714 can be labeled with segment identifiers used by physicians, e.g., left frontal lobe.

The brain scan analyzer 110 includes an atlas matcher 712 that registers the coordinate space of the brain scan 104 to the coordinate space of the brain atlas 714 so that the 3D coordinates of the pixels in the brain scan 104 identified by the image analyzer 710 as corresponding to the possible stroke 106 can be mapped to the coordinate space of a reference brain image depicted in the brain atlas 714. For example, the atlas matcher 712 can perform an iterative process in which a similarity is determined between the brain scan 104 and the reference brain image in the brain atlas 714, and in which random changes can be made to the topology of the brain scan 104 until it is substantially similar to the reference brain image in brain atlas 714.

The brain scan analyzer 110 includes a stroke locator 716 that locates the possible stroke 106 by reading the brain atlas 714 via the atlas matcher 712. A brain location read from the brain atlas 714 using registered coordinates from the atlas matcher 712, e.g., left frontal lobe, can then be included in the stroke diagnosis 112.

The image analyzer 710 can include an image classifier that recognizes stroke regions by learning the characteristics of pixels in a brain scan that indicate a possible stroke. The image classifier can be trained using a training set of brain scans depicting different types of strokes and the learned parameters of the image classifier can be refined using feedback, e.g., feedback provided by the specialist 608. The patterns recognized by the image classifier of the image analyzer 710 along with the location of the possible stroke 106 by the stroke locator 716 can provide the parameters for the stroke diagnosis 112.

In one or more embodiments, the brain scan analyzer 110 can use any known computer image analysis technique or any combination of known computer image analysis techniques. For example, the brain scan analyzer 110 can use computer image analysis techniques based on image intensity, computer image analysis techniques based on pattern recognition, computer image analysis techniques based on 3D object shape, computer image analysis techniques based on machine learning, etc.

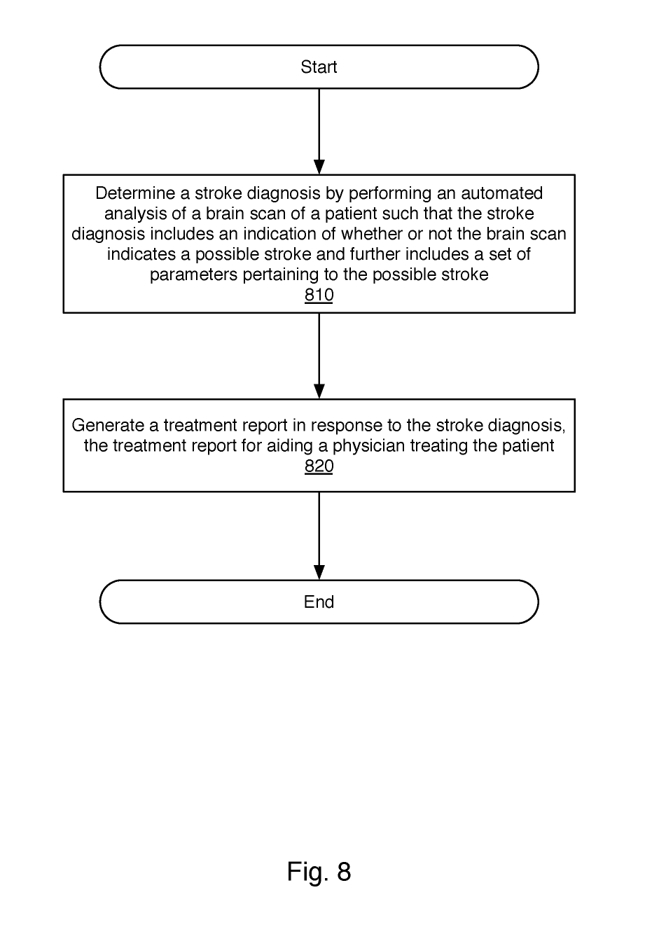

FIG. 8 illustrates a method for stroke treatment support in one or more embodiments. While the various steps in this flowchart are presented and described sequentially, one of ordinary skill will appreciate that some or all of the steps can be executed in different orders and some or all of the steps can be executed in parallel. Further, in one or more embodiments, one or more of the steps described below can be omitted, repeated, and/or performed in a different order. Accordingly, the specific arrangement of steps shown in FIG. 8 should not be construed as limiting the scope of the invention.

At step 810, a stroke diagnosis is determined by performing an automated analysis of a brain scan of a patient such that the stroke diagnosis includes an indication of whether or not the brain scan indicates a possible stroke and further includes a set of parameters pertaining to the possible stroke. The parameters can identify a possible stroke in terms commonly used in the medical profession, e.g., in terms of a brain atlas.

At step 820, a treatment report is generated in response to the stroke diagnosis. The treatment report can aid a physician treating the patient. The treatment report can be generated by accessing a knowledge store. The treatment report can be adapted to the stroke diagnosis from step 810. A knowledge store accessed at step 820 can be a local knowledge store, e.g., in an imaging facility of a hospital, clinic, etc., or a remote knowledge store, e.g., a cloud-based knowledge store.

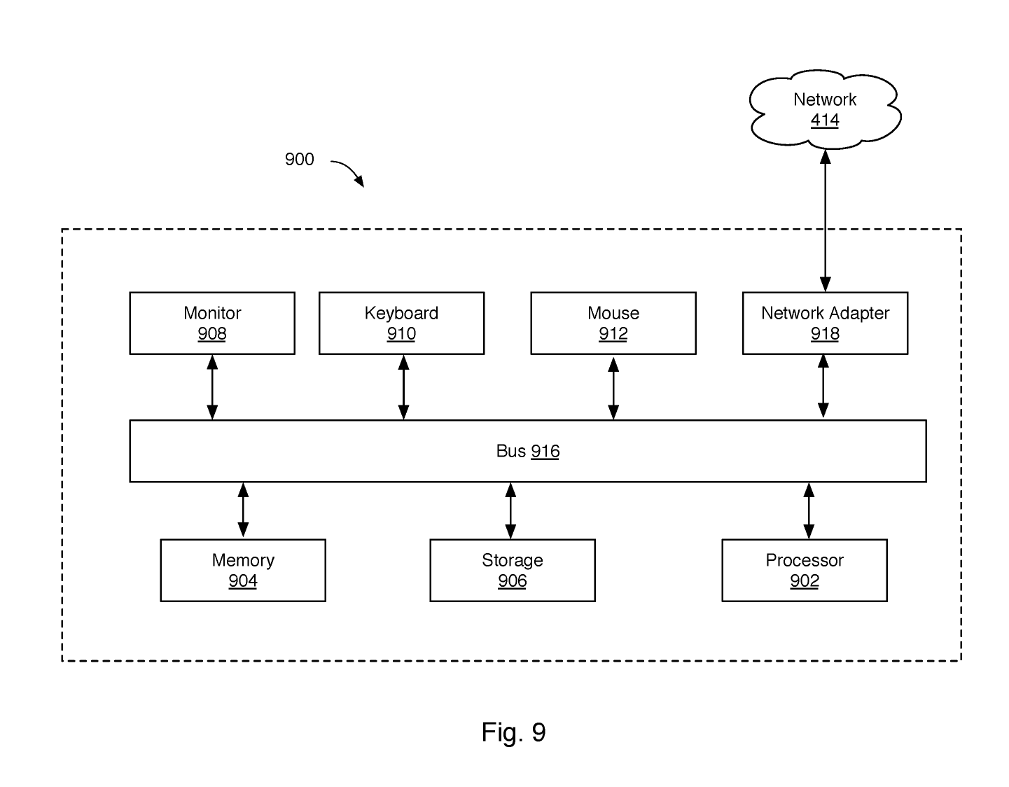

FIG. 9 illustrates a computing system 900 upon which portions of the automated stroke detection and treatment support system 100 can be implemented. The computing system 900 includes one or more computer processor(s) 902, associated memory 904 (e.g., random access memory (RAM), cache memory, flash memory, etc.), one or more storage device(s) 906 (e.g., a hard disk, an optical drive such as a compact disk (CD) drive or digital versatile disk (DVD) drive, a flash memory stick, etc.), a bus 916, and numerous other elements and functionalities. The computer processor(s) 902 may be an integrated circuit for processing instructions. For example, the computer processor(s) may be one or more cores or micro-cores of a processor. The computing system 900 may also include one or more input device(s), e.g., a touchscreen, keyboard 910, mouse 912, microphone, touchpad, electronic pen, or any other type of input device. Further, the computing system 900 may include one or more monitor device(s) 908, such as a screen (e.g., a liquid crystal display (LCD), a plasma display, touchscreen, cathode ray tube (CRT) monitor, projector, or other display device), external storage, input for an electric instrument, or any other output device. The computing system 900 may be connected to the network 414 (e.g., a local area network (LAN), a wide area network (WAN) such as the Internet, mobile network, or any other type of network) via a network adapter 918.

While the foregoing disclosure sets forth various embodiments using specific diagrams, flowcharts, and examples, each diagram component, flowchart step, operation, and/or component described and/or illustrated herein may be implemented, individually and/or collectively, using a range of processes and components.

The process parameters and sequence of steps described and/or illustrated herein are given by way of example only. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While the invention has been described with respect to a limited number of embodiments, those skilled in the art, having benefit of this disclosure, will appreciate that other embodiments may be devised which do not depart from the scope of the invention as disclosed herein.

What is claimed is:

1. An automated stroke detection and treatment support system, comprising:
a brain scan analyzer for determining a stroke diagnosis in response to a brain scan of a patient, the stroke diagnosis including an indication of whether or not the brain scan indicates a possible stroke and a set of parameters pertaining to the possible stroke; and
a treatment planner for generating a treatment report in response to the stroke diagnosis, the treatment report for aiding a physician treating the patient.

2. The automated stroke detection and treatment support system of claim 1, wherein the treatment report identifies a set of expected symptoms for confirming the possible stroke.

3. The automated stroke detection and treatment support system of claim 1, wherein the treatment report identifies a recommended treatment for the possible stroke.

4. The automated stroke detection and treatment support system of claim 1, wherein the treatment planner obtains a set of expected symptoms for confirming the possible stroke for inclusion in the treatment report from a knowledge store holding multiple sets of expected symptoms.

5. The automated stroke detection and treatment support system of claim 1, wherein the treatment planner obtains a recommended treatment for the possible stroke for inclusion in the treatment report from a knowledge store holding multiple recommended treatments.

6. The automated stroke detection and treatment support system of claim 1, further comprising an interface that enables the physician to access the treatment report using a mobile device.

7. The automated stroke detection and treatment support system of claim 1, wherein the treatment report includes a set of text.

8. The automated stroke detection and treatment support system of claim 1, wherein the treatment report includes an illustration.

9. The automated stroke detection and treatment support system of claim 1, wherein the treatment report includes a uniform resource locator.

10. The automated stroke detection and treatment support system of claim 1, further comprising an interface that enables a specialist to provide a feedback regarding the stroke diagnosis.

11. A method for automated stroke detection and treatment support, comprising:
determining a stroke diagnosis by performing an automated analysis of a brain scan of a patient such that the stroke diagnosis includes an indication of whether or not the brain scan indicates a possible stroke and further includes a set of parameters pertaining to the possible stroke; and
generating a treatment report in response to the stroke diagnosis, the treatment report for aiding a physician treating the patient.

12. The method of claim 11, wherein generating a treatment report includes identifying a set of expected symptoms for confirming the possible stroke.

13. The method of claim 11, wherein generating a treatment report includes identifying a recommended treatment for the possible stroke.

14. The method of claim 11, wherein generating a treatment report comprises obtaining a set of expected symptoms for confirming the possible stroke from among multiple sets of expected symptoms each adapted to a particular parameter in the stroke diagnosis.

15. The method of claim 11, wherein generating a treatment report comprises obtaining a recommended treatment for the possible stroke from among multiple recommended treatments each adapted to a particular parameter in the stroke diagnosis.

16. The method of claim 11, further comprising generating an interface that enables a physician responsible for treating the patient to access the treatment report using a mobile device.

17. The method of claim 11, wherein generating a treatment report comprises providing a set of text in the treatment report.

18. The method of claim 11, wherein generating a treatment report comprises providing an illustration in the treatment report.

19. The method of claim 11, wherein generating a treatment report comprises providing a uniform resource locator in the treatment report.

20. The method of claim 11, further comprising generating an interface that enables a specialist to provide a feedback regarding the stroke diagnosis.
